(19) United States
(12) Patent Application Publication
(10) Pub. No.: US 2023/0417749 A1
(43) Pub. Date: Dec. 28, 2023

(54) POLYNUCLEOTIDE NANOSTRUCTURES FOR DETECTING VIRAL INFECTIONS AND OTHER DISEASES

(71) Applicants: ATOM BIOWORKS, INC., Cary, NC (US); THE BOARD OF TRUSTEES OF THE UNVERSITY OF ILLINOIS, Urbaba, IL (US)

(72) Inventors: Xing Wang, Urbana, IL (US); Xiaohu Yao, Cary, NC (US); Nicolas Letoumeau, Cary, NC (US)

(21) Appl. No.: 18/037,433

(22) PCT Filed: Nov. 18, 2021
(86) PCT No.: PCT/US21/59919

§ 371 Date: May 17, 2023

Related U.S. Application Data
(60) Provisional application No. 63/115,268, filed on Nov. 18, 2020.

(51) Int. Cl. G01N 33/569; G01N 33/53; G01N 33/543

(52) U.S. Cl. CPC G01N 33/56983; G01N 33/5308; G01N 33/54386; G01N 2333/165; G01N 2469/10

(57) ABSTRACT

The present disclosure relates to polynucleotide nanostructures and techniques that use polynucleotide nanostructures as biomolecular recognition entities for detecting viral infections, e.g. Covid-19, and other disease. For example, an artificial biopolymer complex can include a network of polynucleotides including structural units connected to one another via a series of arms and junctions, e.g. in the form of a DNA Star. Intersections of three or more arms form the junctions at a predetermined distance from one another. The artificial biopolymer complex further includes binders, e.g. aptamers, attached to the network of polynucleotides that can bind to antigens of a target analyte. The binders are attached at loci on one or more of the arms forming the junctions. The loci are separated by predetermined inter¬binder distances such that the binders are positioned on the network of polynucleotides in a predetermined two-dimensional or three-dimensional spatial pattern that matches a two-dimensional or three-dimensional spatial pattern of the antigens on the target analyte. The nucleic acid oligonucleotides, e.g. the aptamers, from which the nanostructure is formed may be labelled with fluorophores and/or quenchers to detect the binding to a target.

### PRIORITY CLAIM

This application claims the benefit of and priority to U.S. Provisional Application No. 63/115,268, filed on Nov. 18, 2020, which is hereby incorporated by reference in its entirety for all purposes.

### STATEMENT OF GOVERNMENT SUPPORT

The invention was made with government support under Award No. 2027778 awarded by the National Science Foundation (NSF). The government has certain rights in the invention.

### FIELD

The present disclosure relates to detection of analytes, and in particular to polynucleotide nanostructures and techniques that use polynucleotide nanostructures as bimolecular recognition entities for detecting viral infections and other diseases.

### BACKGROUND

Diagnostic tests are used to detect current, active infections or diseases caused by various pathogens (e.g., viruses, bacteria, fungi, protozoa, etc.) such as the severe acute respiratory syndrome coronavirus 2 (SARS-CoV-2). Diagnostic tests can be antigen based, which look for biomarkers on a surface of the pathogen, or they can be molecular based, which look for genomic material specific to the pathogen. In the specific instance of viruses, a host is required to replicate. The virus hijacks the host's cells to produce more viral copies of itself. The genomic material for viruses is deoxyribonucleic acid (DNA) or ribonucleic acid (RNA), which remains in the body while the virus is still replicating and reproducing. Diagnostic tests look for evidence of this replication process to diagnose an active infection of a virus.

Antigen diagnostic tests detect structural features including protein markers on the surface of the virus that may be present in a patient's sample. In contrast, molecular diagnostic tests amplify bits of viral DNA or RNA so that the viral infection can be detected using a specialized test (e.g., PCR, LAMP, CRISPR) capable of detecting viral DNA or RNA. Antigen and molecular tests require samples—such as nasopharyngeal surface cells or sputum/saliva—that are likely to contain the virus. Viruses and other pathogens may also be detected in feces, urine, or blood. For respiratory-presenting diseases like coronavirus disease 2019 (COVID-19) caused by SARS-CoV-2, most tests available or in development use samples from a person's nose (using either nasopharyngeal swabs or anterior nasal swabs) or mouth (using saliva collection cups) to make testing easier for both healthcare providers and patients.

### SUMMARY

Provided herein, according to various embodiments, is an artificial biopolymer complex comprising: a network of polynucleotides comprising structural units connected to one another via a series of arms and junctions, wherein: each of the structural units have a predetermined shape defined by one or more strands of polynucleotides; at least a portion of the one or more strands of polynucleotides of each structural unit is complementary to at least a portion of the one or more strands of polynucleotides of another structural unit, and the complementary portions of the strands of the polynucleotides are hybridized to connect the structural units; the complementary portions of the strands of the polynucleotides form the arms with a predetermined length; and intersections of three or more arms form the junctions at a predetermined distance from one another based on the predetermined length of the arms; and binders attached to the network of polynucleotides, where: the binders bind to antigens of a target analyte; and the binders are attached at loci on one or more of the arms forming the junctions, where the loci are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte.

In some embodiments, each of the antigens comprises one or more epitopes; the binders are arranged in sets of clustered binders; each binder of a set of clustered binders is attached to one of the three or more arms that form a junction; and the binders of each of the sets of clustered binders are attached to the arms at loci that are a predetermined distance from the junction, where the loci are separated by predetermined intra-binder distances such that each set of clustered binders are positioned on the network of polynucleotides in a predetermined two-dimensional or three-dimensional spatial pattern that matches a two-dimensional or three-dimensional spatial pattern of the one or more epitopes on an antigen.

In some embodiments, the junctions are formed by at least 2N arms extending therefrom, and where N is at least 2.

In some embodiments, each of the junctions are formed by at least N arms extending therefrom, and where N is at least 3.

In some embodiments, N binders are attached to the arms that form each of the junctions, and where N is at least 1.

In some embodiments, N is at least 2, and where the N binders are attached to alternating arms that form each of the junctions.

In some embodiments, the two-dimensional or three-dimensional spatial pattern of the antigens is defined by intermolecular spacing of the antigens on a surface of the target analyte.

In some embodiments, the predetermined inter-binder distances of the loci of the binders match the intermolecular spacing of the antigens such that the binders align spatially with the antigens on the surface of the target analyte.

In some embodiments, each of the antigens is (i) a length and width in angstroms or nanometers from other antigens on the target analyte or (ii) a length, width, and depth in angstroms or nanometers from the other antigens on the target analyte, which define the intramolecular spacing of the antigens on the target analyte; each of the binders is (i) a length and width in angstroms or nanometers from other binders on the network of polynucleotides or (ii) a length, width, and depth in angstroms or nanometers from the other binders on the network of polynucleotides, which defines the predetermined inter-binder distances of the loci of the binders; and the predetermined inter-binder distances of the loci of the binders match the intermolecular spacing of the antigens such that the binders align spatially with the antigens on the surface of the target analyte.

In some embodiments, the two-dimensional or three-dimensional spatial pattern of the one or more epitopes is defined by intramolecular spacing of the one or more epitopes on a surface of the antigen.

In some embodiments, the predetermined intra-binder distances of the loci of the binders of each of the sets of clustered binders match the intramolecular spacing of the one or more epitopes such that the sets of clustered binders align spatially with the one or more epitopes on the surface of the antigens.

In some embodiments, each of the epitopes is (i) a length and width in angstroms or nanometers from other epitopes on the antigen or (ii) a length, width, and depth in angstroms or nanometers from the other epitopes on the antigen, which define the intramolecular spacing of the one or more epitopes on the antigen; each of the binders of each of the sets of clustered binders is (i) a length and width in angstroms or nanometers from other binders of each of the sets of clustered binders on the network of polynucleotides or (ii) a length, width, and depth in angstroms or nanometers from the other binders of each of the sets of clustered binders on the network of polynucleotides, which defines the predetermined intra-binder distances of the loci of the binders of each of the sets of clustered binders; and the predetermined intra-binder distances of the loci of the binders of each of the sets of clustered binders match the intermolecular spacing of the epitopes such that each of the sets of clustered binders align spatially with the one or more epitopes on the surface of the antigens.

In some embodiments, each of the structural units have the same predetermined shape defined by the one or more strands of polynucleotides.

In some embodiments, the network of polynucleotides has a length L and a width W defined by a number S of structural units, and where L is 1 or more and W is 1 or more.

In some embodiments, L is 2 between 2 and 5 and W is between 2 and 5.

In some embodiments, the predetermined shape is a rhombus, a triangle, a pentagon, or a hexagon.

In some embodiments, the one or more strands of polynucleotides are single stranded DNA, and the arms are double stranded DNA.

In some embodiments, the target analyte is severe acute respiratory syndrome coronavirus 2 (SARS-CoV-2), and the antigens comprise trimeric spike glycoproteins.

In some embodiments, each of the binders is an aptamer, an antibody, a peptide, a nanobody, an antibody mimic, or a small analyte ligand.

In some embodiments, the artificial biopolymer complex further comprises locking molecules that attach each of the binders to the network of polynucleotides.

In some embodiments, the locking molecules comprise a single stranded chain of nucleic acids hybridized to form a portion of the arms attached to the binders.

In some embodiments, the artificial biopolymer complex further comprises quenchers attached to the locking molecules and fluorophores attached to the binders.

In some embodiments, the artificial biopolymer complex further comprises quenchers attached to the binders and fluorophores attached to the locking molecules.

In some embodiments, the artificial biopolymer complex further comprises quenchers attached to the network of polynucleotides and fluorophores attached to the binders.

In some embodiments, the artificial biopolymer complex further comprises quenchers attached to the binders and fluorophores attached to the network of polynucleotides.

In some embodiments, the artificial biopolymer complex further comprises quenchers and fluorophores attached to the binders.

In various embodiments, a method is provided for determining a presence or absence of a target analyte in a sample. The method comprises: obtaining the artificial biopolymer complex of any of the embodiments described herein; adding the artificial biopolymer complex to the sample; detecting a signal from the sample; and determining the presence or absence of the target analyte in the sample based on the signal.

In some embodiments, the determining is a qualitative or quantitative determination based on the signal.

In some embodiments, the signal is detected over a detection period of time to identify a rate of change of the signal during the detection period of time, and where the rate of change above a threshold is indicative of the presence of the target analyte.

In some embodiments, the detection period of time is about 100 seconds in length.

In some embodiments, the detection period of time is from about 30 seconds to 10 minutes in length.

In some embodiments, the signal is a fluorescent signal.

In some embodiments, the method further comprises: binding the artificial biopolymer complex to the target analyte; in response to the binding, releasing one or more of the quenchers from the locking molecules, the network of polynucleotides, or the binders; and in response to the release of the one or more quenchers, generating the fluorescent signal by one or more fluorophores that are no longer quenched by the one or more quenchers.

In some embodiments, the method further comprises: binding the artificial biopolymer complex to the target analyte; in response to the binding, changing a conformation of the binders attached to the antigens of the target analyte or the epitopes of the antigens of the target analyte; in response to the conformation change to the binders, reducing quenching of the fluorescent signal by one or more of the quenchers; and in response to reducing the quenching, generating the fluorescent signal by one or more fluorophores that are no longer quenched by the one or more quenchers.

In various embodiments, a method is provided for determining a presence or absence of a target analyte in a sample. The method comprises: obtaining the artificial biopolymer complex of any of the embodiments disclosed herein; adding the artificial biopolymer complex to the sample; adding quenchers to the sample; detecting a signal from the sample; and determining the presence or absence of the target analyte in the sample based on the signal.

In some embodiments, the quenchers are attached to oligonucleotides structured to attach to the binders; fluorophores are attached to the locking molecules, the network of polynucleotides, or the binders; and the signal is a fluorescent signal.

In some embodiments, the method further comprises: binding the artificial biopolymer complex to the target analyte; binding the quenchers to one or more binders that do not attach to the antigens of the target analyte or the epitopes of the antigens of the target analyte; and in response to the binding of the quencher, quenching the fluorescent signal by one or more fluorophores attached to the locking molecules, the network of polynucleotides, or the binders.

In some embodiments, the method further comprises: prior to adding the quenchers to the sample, incubating the sample with the artificial biopolymer complex for a first predetermined amount of time; after the incubating for the first predetermined amount of time and prior to adding the quenchers to the sample, detecting the signal from the sample to obtain a first reading; and prior to detecting the signal from the sample, incubating the sample with the artificial biopolymer complex and the quenchers for a second predetermined amount of time, where the detecting the signal from the sample after adding the quenchers obtains a second reading, and the presence or absence of the target analyte in the sample is determined based on the first reading and the second reading.

In some embodiments, the signal is detected over a detection period of time to identify a rate of change of the signal during the detection period of time, and where the rate of change above a threshold is indicative of the absence of the target analyte.

In various embodiments, a method is provided for treating a subject. The method comprising: obtaining the artificial biopolymer complex of any of the embodiments disclosed herein; and administering the artificial biopolymer complex to the subject in an amount sufficient to provide a treatment effect.

In some embodiments, the treatment effect is a prophylactic effect or a therapeutic effect.

In some embodiments, the artificial biopolymer complex further comprises one or more therapeutic agents attached to the network of polynucleotides.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 illustrates an artificial biopolymer complex that includes a network of polynucleotides as a recognition entity for the detection of antigens according to various embodiments of the present disclosure.

FIG. 2 illustrates another artificial biopolymer complex that a network of polynucleotides as a recognition entity for the detection of antigens according to various embodiments of the present disclosure.

FIG. 3 illustrates another artificial biopolymer complex that includes a network of polynucleotides as a recognition entity for the detection of antigens according to various embodiments of the present disclosure.

FIG. 4A illustrates networks of polynucleotides with different numbers of structural units according to various embodiments of the present disclosure.

FIG. 4B illustrates the networks of polynucleotides characterized by 1% agarose gel electrophoresis (AGE) in 1×TA-Mg²⁺ buffer according to various embodiments of the present disclosure.

FIG. 4C illustrates atomic force microscopy images (AFM) showing the networks of polynucleotides according to various embodiments of the present disclosure.

FIG. 5 illustrates a locking molecule hybridized to a binder according to various embodiments of the present disclosure.

FIG. 6 illustrates pairs of binders and locking molecules for binding to epitopes of an antigen according to various embodiments of the present disclosure.

FIG. 7 illustrates quenchers added to a sample of networks of polynucleotides bound to an antigen according to various embodiments of the present disclosure.

FIG. 8 illustrates a quencher and a fluorophore attached to a network of polynucleotides for binding to epitopes of an antigen according to various embodiments of the present disclosure.

FIG. 9 shows the results of a fluorescence-based assay using a network of polynucleotides designed to bind to SARS-CoV-2 virions according to various embodiments of the present disclosure.

FIG. 10A shows the results of a fluorescence-based assay using a network of polynucleotides designed to bind to SARS-CoV-2 virions in a saliva sample at different virus concentrations according to various embodiments of the present disclosure.

FIG. 10B shows the results of a fluorescence-based assay using a network of polynucleotides designed to bind to SARS-CoV-2 virions in a control sample at different virus concentrations according to various embodiments of the present disclosure.

FIGS. 11A-11E show the results of binding data for various artificial biopolymer complexes according to various embodiments of the present disclosure.

FIG. 12 illustrates a process for determining a presence or absence of a target analyte in a sample according to various embodiments of the present disclosure.

FIG. 13 illustrates a process for determining a presence or absence of a target analyte in a sample according to various embodiments of the present disclosure.

FIG. 14 illustrates a process for treating a subject using an artificial biopolymer complex according to various embodiments of the present disclosure.

### DETAILED DESCRIPTION

The details of various embodiments of the invention are set forth in the description below. Other features, objects, and advantages of the invention will be apparent from the description and the drawings, and from the claims.

### Overview

Disclosed herein are polynucleotide nanostructures (also referred to herein as polynucleotide scaffolds) and techniques that use polynucleotide nanostructures as recognition entities for the detection of target analytes. The design of the polynucleotide nanostructures takes advantage of a polyvalent binding strategy to bind to a target molecule with a high binding avidity. This enables targeted detection with high sensitivity and specificity, and therapy via the introduction of toxins/therapeutics to the target analytes or preventing entry of a pathogen into host cells.

Conventional viral infection or disease detection methods make use of antibodies to detect the presence or absence of a target analyte. But, antibodies by themselves suffer from several limitations in diagnostic use. Because antibodies are produced by biological processes in animals or bacteria, they are expensive, time consuming to develop, and their qualities can vary between batches. Additionally, antibodies are proteins that are prone to denature, so antibodies are unstable for use in many environmental conditions, and are not viable after long-term storage. Another limitation of methods that use antibodies by themselves, is their sensitivity and specificity, or rate of detecting the target analyte correctly, which means a potential for a high rate of false negatives.

Moreover, conventional polynucleotide nanostructure-based detection mechanisms typically rely on surface proteins that are rigidly fixed in position on the surface of a target analyte. For example, in Dengue and Zika viruses, the rigidity of the viral capsid can be leveraged to design conventional polynucleotide nanostructure-based detection mechanisms and facilitate detection. However, for membrane-containing viruses or cells such as SARS-CoV-2, HIV, and influenza, where surface proteins have greater mobility and the membranes lack the rigidity of capsid viruses, conventional polynucleotide nanostructure-based detection mechanisms lack the capability to bind to the surface proteins with high binding avidity.

To address these limitations and others, the polynucleotide nanostructures or scaffolds of the present disclosure make use of a network of polynucleotides that provide a structure for a defined spacing of binding ligands (e.g., aptamers) to bind specifically to antigen clusters on the outer surface of the membrane of a target analyte, such as a membrane-containing virus. The defined spacing includes (i) inter-antigen spacing according to target antigens on an analyte such as the surface of an encapsulated biological entity, and/or (ii) intra-antigen spacing according to target epitopes on an antigen such as a multimeric surface protein or other multimeric target molecule. Advantageously, the defined inter-antigen and intra-antigen spacing allows for the polynucleotide nanostructures to be constructed to bind specifically to multiple targets on the surface of an analyte, where the targets are mobile within and/or on the surface (e.g., have a probability of being located within an area envelope extending around a central average position), such as on a viral or cell membrane. This specific binding of the polynucleotide nanostructures to antigen clusters increases sensitivity and specificity of detection of the analyte.

One illustrative embodiment of the present disclosure is directed to an artificial biopolymer complex that includes a network of polynucleotides comprising structural units connected to one another via a series of arms and junctions. Each of the structural units have a predetermined shape defined by one or more strands of polynucleotides. At least a portion of the one or more strands of polynucleotides of each structural unit is complementary to at least a portion of the one or more strands of polynucleotides of another structural unit, and the complementary portions of the strands of the polynucleotides are hybridized to connect the structural units. The complementary portions of the strands of the polynucleotides form the arms with a predetermined length. Intersections of three or more arms form the junctions at a predetermined distance from one another based on the predetermined length of the arms. Binders are attached to the network of polynucleotides. The binders bind to antigens of a target analyte. The binders are attached at loci on one or more of the arms forming the junctions, where the loci are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte.

### Artificial Biopolymer Complexes

The artificial biopolymer complexes described herein provide a polynucleotide nanostructure to support defined spacing for binders that bind to a target antigen. In some instances, the loci of the binders are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte. The two-dimensional or three-dimensional spatial pattern of the antigens is defined by intermolecular spacing of the antigens on a surface of the target analyte (e.g., a cluster of antigens or clusters of antigens). In some instances, the loci are separated by predetermined intra-binder distances such that each set of clustered antigen binders are positioned on the network of polynucleotides in a predetermined two-dimensional or three-dimensional spatial pattern that matches a two-dimensional or three-dimensional spatial pattern of the one or more epitopes on an antigen. The two-dimensional or three-dimensional spatial pattern of the one or more epitopes is defined by intramolecular spacing of the one or more epitopes on a surface of the antigen.

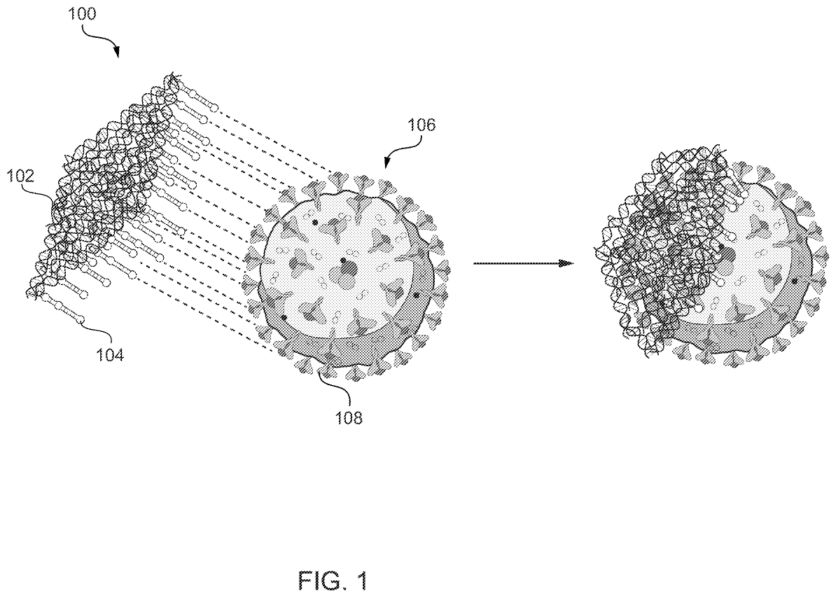

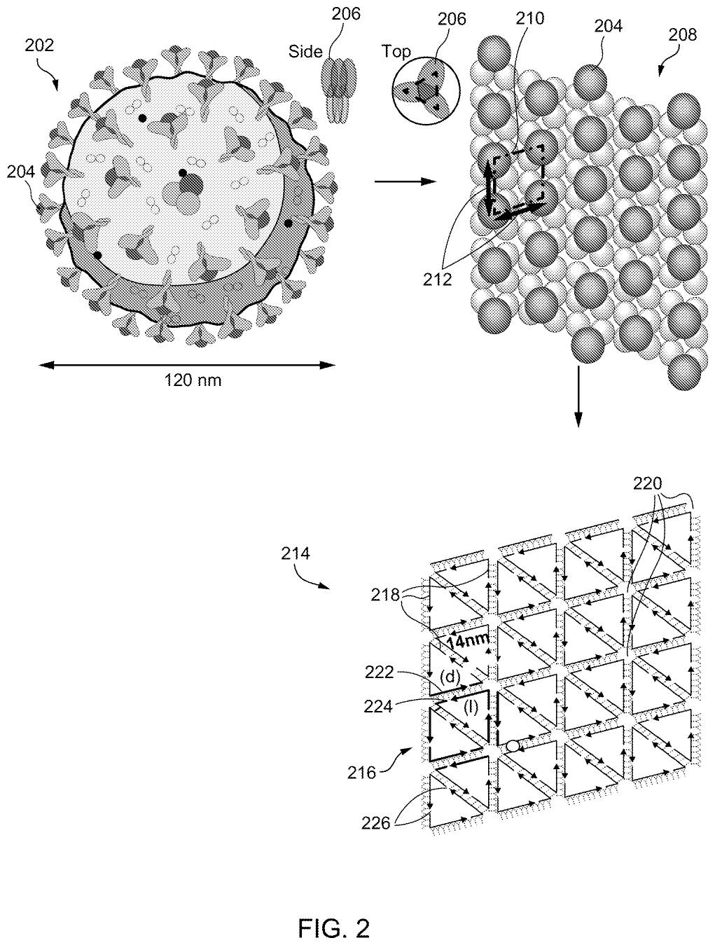

FIG. 1 illustrates an artificial biopolymer complex 100 that use polynucleotide nanostructures as biomolecular recognition entities for the detection of antigens according to various embodiments of the present disclosure. The artificial biopolymer complex 100 comprises a network of polynucleotides 102 and binders 104 attached to the network of polynucleotides 102. The binders 104 of the artificial biopolymer complex 100 can bind to antigens 108 of a target analyte 106. As shown in FIG. 2, the network of polynucleotides 214 (e.g., the network of polynucleotides 102) comprise structural units 216 connected to one another via a series of arms 218 and junctions 220. Each of the structural units 216 have a predetermined shape (e.g., a triangle or rhombus) defined by one or more strands of polynucleotides. The one or more strands of polynucleotides may be single stranded DNA or RNA (ssDNA or ssRNA). At least a portion 222 of the one or more strands of polynucleotides of each structural unit is complementary to at least a portion 224 of the one or more strands of polynucleotides of another structural unit, and the complementary portions of the strands of the polynucleotides are hybridized to connect the structural units. Consequently, the arms 218 or at least a portion thereof may be double stranded DNA or RNA. The complementary portions of the strands of polynucleotides form the arms 218 with a predetermined length (l), and the intersections of the three or more arms 218 form the junctions 220 at a predetermined distance (d) from one another based on the predetermined length (l) of the arms 218.

The network of polynucleotides 214 provide addressable anchor loci 226 for displaying the same or different binders 104 at each anchor location 226. The anchor loci 226 may be located on one or more of the three or more arms 218 that form a junction 220. The network of polynucleotides 214 is functionalized by attaching the binders 104 at the addressable loci 226 on various surfaces of the network of polynucleotides 214.

In some instances, the loci 226 are separated by predetermined inter-binder distances such that the binders 104 are positioned on the network of polynucleotides 214 in a predetermined two-dimensional or three-dimensional spatial pattern that matches a two-dimensional or three-dimensional spatial pattern of the antigens 108 on the target analyte 106. For example, FIG. 2 also depicts a target analyte 202 (e.g., target analyte 106) that may be severe acute respiratory syndrome coronavirus 2 (SARS-CoV-2) having a diameter of approximately 120 nm, and antigens 204 (e.g., antigens 108) that may be trimeric spike glycoproteins (TS-p). The antigens 204 may comprise one or more epitopes 206. FIG. 2 depicts a side view and a top view of three epitopes 206 per antigen 204 for the target analyte 202. As used herein, epitope refers generally to the target region of the antigen onto which a binder specifically binds. Thus, for a trimeric protein with an epitope on each subunit of the trimer, a corresponding set of three binders can bind to the trimeric protein with high specificity. The surface 208 of the target analyte 202 comprises a two-dimensional or three-dimensional spatial pattern 210 that is defined by intermolecular spacing 212 of the antigens 204. The predetermined inter-binder distances of the loci 226 of the binders 104 match the intermolecular spacing of the antigens 204 such that the binders 104 align spatially with the antigens 204 on the surface of the target analyte 202.

More specifically, each of the antigens 204 is (i) a length and width in angstroms or nanometers from other antigens on the target analyte 202 or (ii) a length, width, and depth in angstroms or nanometers from the other antigens on the target analyte 202, which define the intermolecular spacing of the antigens 204 on the target analyte 202. Each of the binders 104 is (i) a length and width in angstroms or nanometers from other binders on the network of polynucleotides 214 or (ii) a length, width, and depth in angstroms or nanometers from the other binders on the network of polynucleotides 214, which defines the predetermined inter-binder distances of the loci 226 of the binders 104. The predetermined inter-binder distances of the loci 226 of the binders 104 match the intermolecular spacing of the antigens 204 such that the binders 104 align spatially with the antigens 204 on the surface of the target analyte 202.

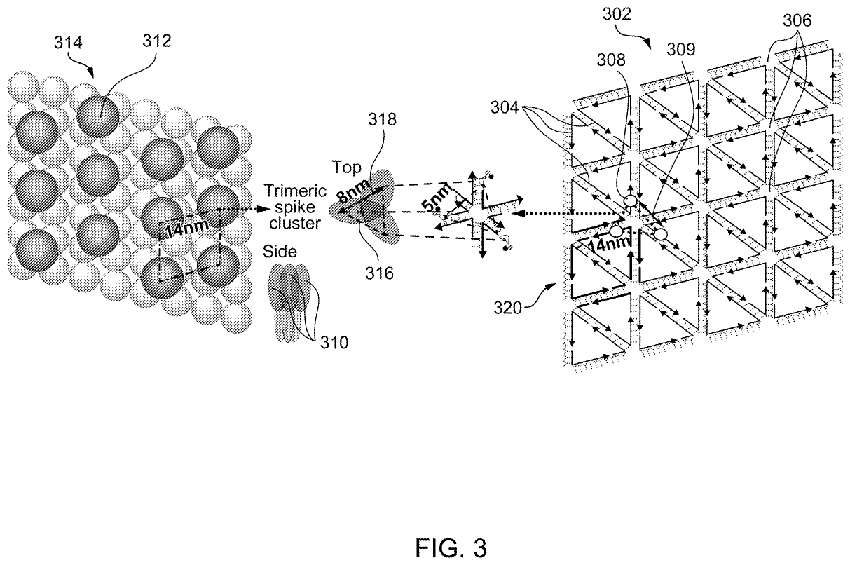

In some instances, as shown in FIG. 3, the binders 104 are arranged on the network of polynucleotides 302 (e.g., network of polynucleotides 214) in sets of clustered binders. The binders 104 of each of the sets of clustered binders are attached to one or more of the three or more arms 304 that form a junction 306. For instance, each binder 104 may be attached to one of the three or more arms 304 that form a junction 306. The binders 104 of each of the sets of clustered binders are attached to the arms 304 at loci 308 that are a predetermined distance from the junction 306. The loci 308 are separated by predetermined intra-binder distances 309 such that each set of clustered binders are positioned on the network of polynucleotides in a predetermined two-dimensional or three-dimensional spatial pattern that matches a two-dimensional or three-dimensional spatial pattern 316 of the one or more epitopes 310 on an antigen 312. The two-dimensional or three-dimensional spatial pattern 316 of the one or more epitopes 310 is defined by intramolecular spacing 318 of the one or more epitopes 310 on the surface 314 of the target analyte 202. The binders 104 are formed on the network of polynucleotides 302 to align with the intramolecular spacing 318 of the epitopes 310 on the antigen 312.

More specifically, each of the epitopes 310 is (i) a length and width in angstroms or nanometers from other epitopes on the antigen 312 or (ii) a length, width, and depth in angstroms or nanometers from the other epitopes on the antigen 312, which define the intramolecular spacing 318 of the epitopes 310 on the antigen 312. Each binder 104 of a set of clustered binders is (i) a length and width in angstroms or nanometers from other binders of the set of clustered binders on the network of polynucleotides 302 or (ii) a length, width, and depth in angstroms or nanometers from the other binders of the set of clustered binders on the network of polynucleotides 302, which defines the predetermined intra-binder distances 309 of the loci 308 of the binders 104 of each of the sets of clustered binders. The predetermined intra-binder distances 309 of the loci 306 of the binders 104 of each of the sets of clustered binders match the intermolecular spacing 318 of the epitopes 310 such that each of the sets of clustered binders align spatially with the one or more epitopes 310 on the surface of the antigens 312. In certain instances, the intramolecular spacing 318 of the epitopes 310 is between 1 nm and 15 nm.

In some instances, the junctions 306 may be formed by at least 2N arms 304 extending from the junction 306. Examples of N can include N being at least 2 or at least 3. In some instances, N binders 104 may be attached to the arms 304 that form each of the junctions 306, with N being at least 1. In some instances, N binders 104 may be attached to the arms 304 at regularly spaced intervals around the junctions 306. In some instances where N is at least 2, N binders 104 may be attached to alternating arms 304 that form each of the junctions 306. Each of the binders 104 can be an aptamer, an antibody, antibody fragment, a peptide, a nanobody, an antibody mimic (e.g., an affimer or a molecularly imprinted polymer), or a small analyte ligand. In instances in which the binders are aptamers, the aptamers may be developed and selected via systematic evolution of ligands by exponential enrichment (SELEX), also referred to as in vitro selection or in vitro evolution. SELEX is a combinatorial chemistry technique in molecular biology for producing oligonucleotides of either ssDNA or ssRNA that specifically bind to a target ligand or ligands.

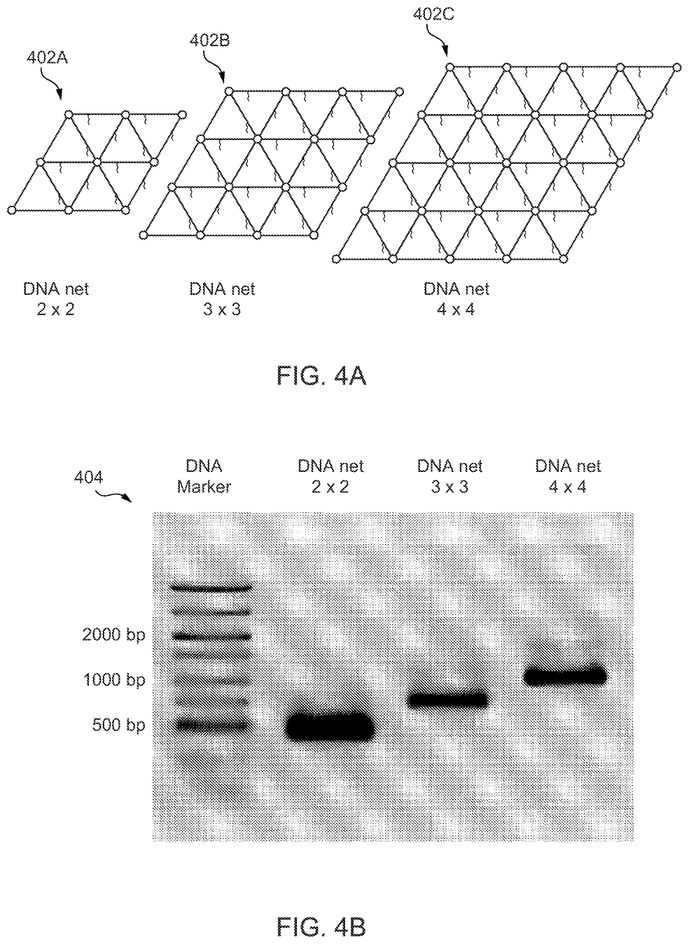

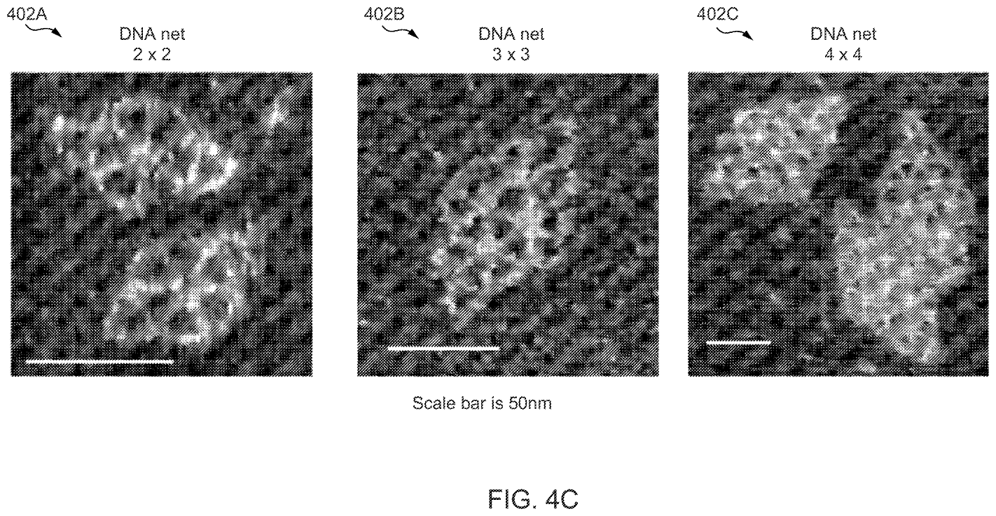

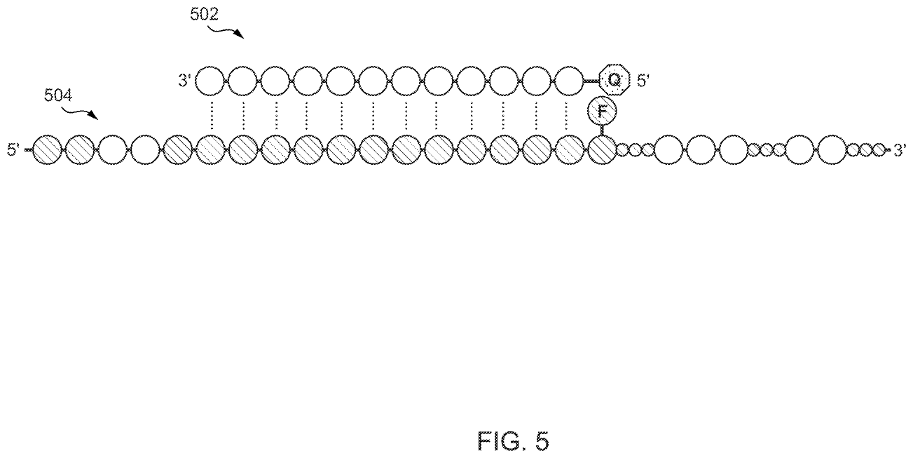

In some instances, each structural unit 320 forming the network of polynucleotides 302 has a same predetermined shape defined by the one or more strands of polynucleotides. Examples of the predetermined shape include a rhombus, a triangle, a pentagon, or a hexagon. The network of polynucleotides 302 can have a length L and a width W defined by a number S of structural units 320, where L can be 1 or more and W can be 1 or more. FIG. 4A illustrates networks of polynucleotides 402A-C with different numbers of structural units according to various embodiments of the present disclosure. For the network of polynucleotides 402A, S is 4, L is 2, and W is 2. For the network of polynucleotides 402B, S is 9, L is 3, and W is 3. For the network of polynucleotides 402C, S is 16, L is 4, and W is 4. In some instances, L may be between 2 and 5 and W may be between 2 and 5. FIG. 4B illustrates the networks of polynucleotides 402A-C characterized by 1% agarose gel electrophoresis (AGE) 404 in 1×TA-Mg²⁺ buffer according to various embodiments of the present disclosure. As shown, the total number of base pairs for the network of polynucleotides increases as the number of structural units increases. FIG. 4C illustrates atomic force microscopy images (AFM) showing networks of polynucleotides 402A-C according to various embodiments of the present disclosure.

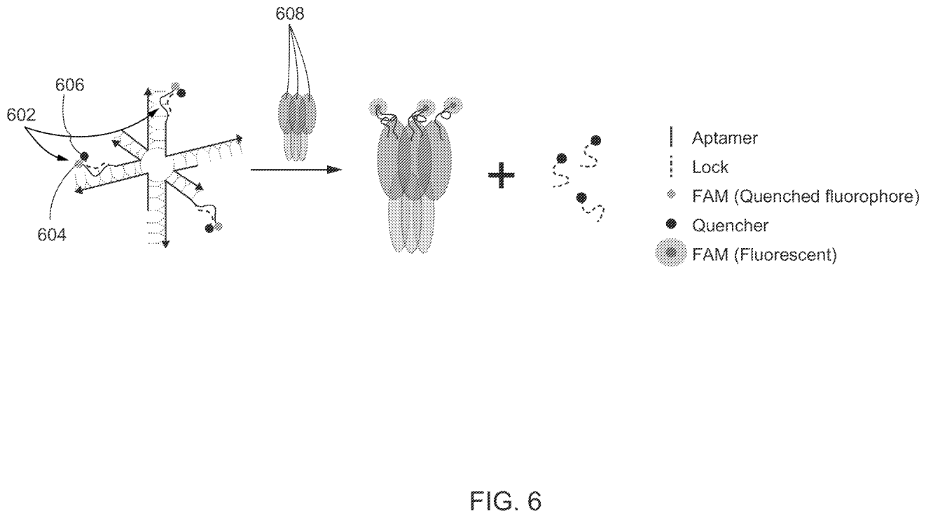

In some instances, the binders 104 are attached to the arms 304 via Van der Waals forces, hydrogen binding, and/or electrostatic forces. In some instances, the binders 104 are attached to the arms 304 via covalent bonds with functional groups on the arms 304. In some instances, the binders 104 are attached to the arms 304 via antibodies, antibody fragments, or nanobodies covalently bonded with functional groups on the arms 304. The covalently bound antibodies, antibody fragments, or nanobodies may target specific regions of the binder such as His-Tags or Fc regions. In other instances, as shown in FIG. 5, locking molecules 502 are used to attach the binders 504 to the arms 304. The locking molecules 502 may be sequences of nucleotides structured with complementary bases to portions of the arms 304 and/or portions of the binders 104 such that the locking molecules 502 can attach to the arms 304 and/or the binders 104 with some degree of affinity. In some instances, the locking molecule 502 is an oligonucleotide or an polynucleotide structured to bind to the arms 304 and/or the binders 504. The locking molecules 502 may be a single stranded chain of nucleic acids hybridized to form a portion of the arms 304 attached to the binders 504. Such binding affinity provides for stability of the network of polynucleotides 302. In addition, for some detection mechanisms described herein that rely on separation of the binders 504 from the locking molecules 502 to generate a signal, such binding affinity optimally is designed or selected to enable separation of the binders 504 and the locking molecules 502 upon binding of the binder 504 to antigens 312 on the target analyte 202, e.g., in view of the binding affinity between the binders 504 and the antigens 312. In some instances, the binders 504 are configured to bind to a target analyte 202 with a higher affinity than the binding interaction between the locking molecules 502 and the binders 504 bound to the network of polynucleotides 302.

In some instances, the network of polynucleotides 302 comprises a signaling mechanism that switches in response to binding of the network of polynucleotides 302 to the target analyte 202. The signaling mechanism may be a combination of reporters (e.g., fluorophores) and quenchers. The efficiency of quenching is substantially distance dependent. For example, if a fluorophore and quencher are far apart, there is fluorescence; if a fluorophore and quencher are close together in space, fluorescence is suppressed. The quenchers may be Dabcyl, Rhodamine, Black Hole Quenchers (BHC), the like, or any combination thereof. The fluorophores may be fluorescein amidites (FAM), the like, or any combination thereof. The reporter and quencher are placed at specific sites on the artificial biopolymer complex such that a change in their distance leveraged from conformational changes occurring during binding will produce a maximal change in fluorescence and effectively signal the event being monitored (e.g., binding of the network of polynucleotides 302 to the target analyte 202). For example, binder 504 conformation change upon binding may cause a reduction in Förster resonance energy transfer (FRET) quenching efficiency or disruption of static quenching as the FAM moves further away from the BHQ. In some instances, the quenchers are attached to the locking molecules 502 and the fluorophores are attached to the binders 504. Alternatively, the fluorophores are attached to the locking molecules 502 and the quenchers are attached to the binders 504. In other examples, the quenchers are attached to the network of polynucleotides 302 and the fluorophores are attached to the binders 504. In some examples, the quenchers are attached to the binders 504 and the fluorophores are attached to the network of polynucleotides 302. Alternatively, the quenchers and the fluorophores are attached to the binders 504.

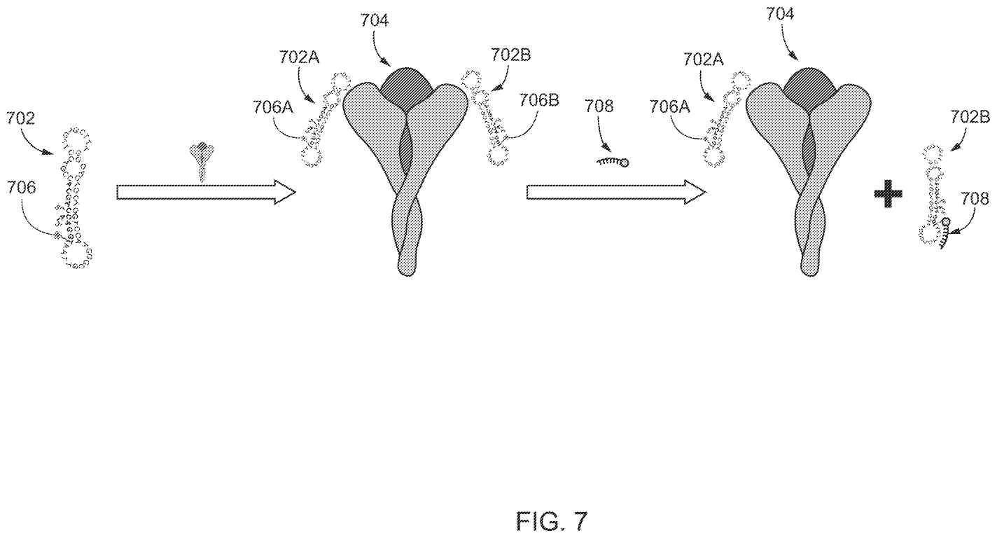

In one exemplary detection mechanism (strand displacement), the quenchers and fluorophores are attached to the binders such that prior to the event being monitored (e.g., binding of the network of polynucleotides 302 to the target analyte 202), the quenchers inhibit the signal of the fluorophores. The quenchers may be attached to the binders via the locking molecules. Upon binding of the network of polynucleotides to the target analyte, the binding affinity of the locking molecules enables separation of the binders and the locking molecules, this separation displaces the quenchers from the fluorophores allowing for the fluorophores to produce a fluorescence signal. For example, FIG. 6 illustrates pairs 602 of binders 604 and locking molecules 606 bound to epitopes 608 (e.g., epitopes 310) of an antigen 312 according to various embodiments of the present disclosure. The pairs 602 may be arranged on a network of polynucleotides 302 to align with the intramolecular spacing of the epitopes 608 on the antigen 312. In FIG. 6, the binders 604 are attached to quenchers and the locking molecules 606 are attached to fluorophores. When the pairs 602 bind with the epitopes 608 of an antigen 312, the pairs 602 may separate due to the binding avidity between the pairs 602 and the epitopes 608 being stronger than the binding avidity between the binders 604 and the locking molecules 606. Separating the binders 604 and the locking molecules 606 can trigger the release of the fluorescence signal that may be detected by a portable fluorimeter or bench top, high throughput microplate reader or RT-PCR system.

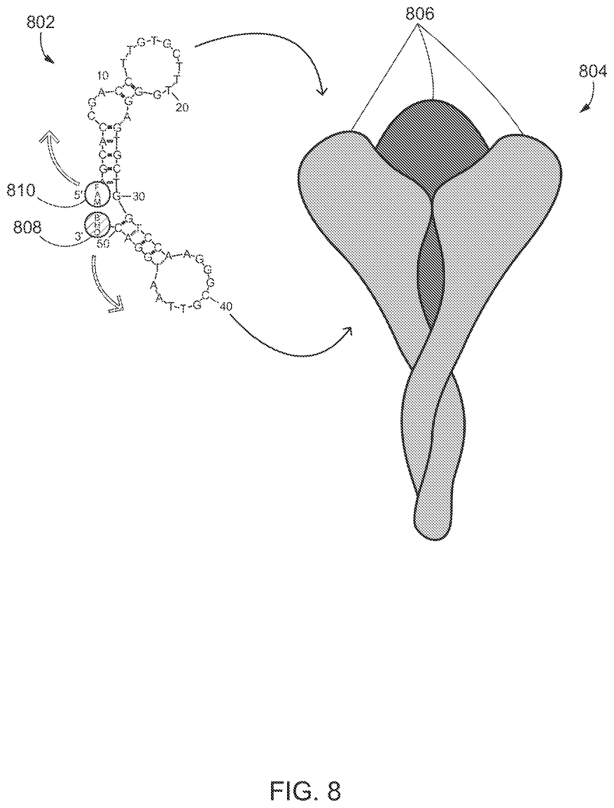

In another exemplary detection mechanism (competitive assay), fluorophores release a fluorescence signal prior to and after the event being monitored (e.g., binding of the network of polynucleotides 302 to the target analyte 202). Fluorophore quenching is inhibited by a presence of antigen bound to the network of polynucleotides. However, the lower the amount of antigen, the less quenching is inhibited, and a corresponding decrease in fluoresce is observable. In some instances, the quenchers are attached to a molecule, e.g., a polynucleotide or oligonucleotide. In other instances, the quencher id attached to the locking molecule, and the locking molecule is attached to a molecule, e.g., a polynucleotide or oligonucleotide. The attachment to the molecule introduces steric hindrance that prevents the quencher from interacting with fluorophores bound to the antigen and only allows the quencher to interact with unbound fluorophores. For example, FIG. 7 illustrates quenchers 708 added to a sample of networks of polynucleotides 702A-B bound to antigen 704 according to various embodiments of the present disclosure. Fluorophores 706A-B are attached to binders of each network of polynucleotides 702. Some of the networks of polynucleotides 702 may become fully saturated with antigen 704 (i.e., substantially all binders have bound to the antigen). The terms “substantially,” “approximately” and “about”, as used herein, are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. Some of the networks of polynucleotides 702 may become partially saturated with antigen 704 (i.e., a smaller percentage, e.g., less than 50%, of the binders have bound to the antigen).

Once the quenchers 708 are added to the sample of networks of polynucleotides 702A-B bound to antigen 704, the quenchers 708 compete with the antigen 704 for binding to the binders. By the quenchers 708 binding to binders, the quenchers 708 can quench the fluorescent signal produced by fluorophores 706A-B (as illustrated 706B). It may be difficult for the quenchers 708 to bind to binders bound to the antigen 704. Therefore, higher amounts of antigens 704 may prevent higher amounts of quenchers 708 from quenching fluorescent signals from fluorophores 706A-B, which may lead to smaller decreases in observed fluorescent signals produced after the quenchers 708 are added to the sample.

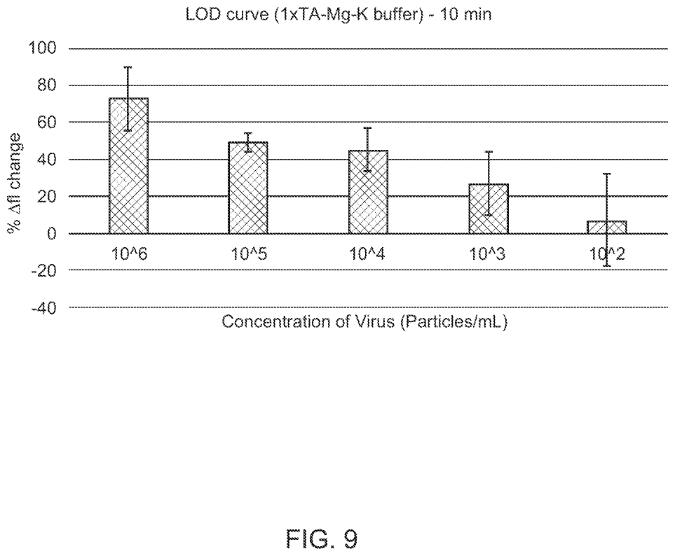

In another exemplary detection mechanism (conformation change), the quenchers and fluorophores are attached to the binders and/or the networks of polynucleotides such that prior to the event being monitored (e.g., binding of the network of polynucleotides 302 to the target analyte 202), the quenchers inhibit the signal of the fluorophores. The quenchers may be attached to the binders and/or the networks of polynucleotides at locations in proximity to locations at which the fluorophores are attached to the binders and/or the networks of polynucleotides. Upon binding of the binders and/or the networks of polynucleotides to the target analyte, the binders and/or the networks of polynucleotides undergo conformational changes. The conformational changes separate the quenchers from the fluorophores allowing for the fluorophores to produce a fluorescence signal. For example, FIG. 8 illustrates quenchers 808 and fluorophores 810 attached to a network of polynucleotides 802 for binding to epitopes 806 of an antigen 804 according to various embodiments of the present disclosure. In some instances, the quenchers 808 and the fluorophores 810 may be attached to binders on the network of polynucleotides 802. The quenchers 808 and the fluorophores 810 may be in close proximity such that the quenchers 808 quench a fluorescent signal generated by the fluorophores 810. In some instances, the quenchers 808 and the fluorophores 810 may be located between 0.5 and 1 nm apart in length on the network of polynucleotides 802 and/or binders. As the binders of the network of polynucleotides 802 bind to the antigen 804, or bind to the epitopes 806 of the antigen 804, the network of polynucleotides 802 and/or the binders may experience conformational changes. The conformational changes can include the quenchers 808 and the fluorophores 810 moving away from one another. In some instances, the quenchers 808 and the fluorophores 810 may move farther than 1 nm of length apart. In response to the conformational changes, the quenchers 808 may reduce quenching of the fluorescent signal produced by the fluorophores 810. In some instances, a higher concentration of antigens 804 may lead to faster generation of fluorescent signals, and stronger signal strength of fluorescent signals. Likewise, a lower concentration of antigens 804 may lead to slower generation of fluorescent signals, and weaker signal strength of fluorescent signals.

### EXAMPLES

The artificial biopolymer complexes and techniques implemented in various embodiments may be better understood by referring to the following examples. Although, these examples are specific to SARS-CoV-2 virions, it should be understood that artificial biopolymer complexes and techniques as described herein may be applicable to any virus or other pathogen.

### Example 1: Proof of Concept Fluorescence Based Assay

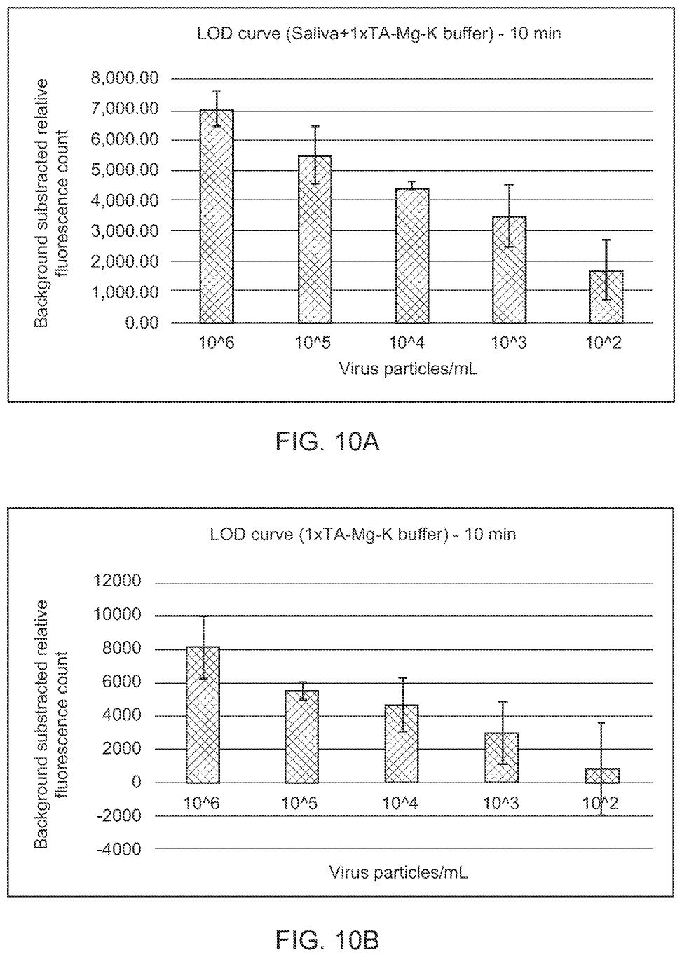

As shown in FIG. 9, a fluorescence-based assay using a network of polynucleotides designed to bind to SARS-CoV-2 virions was successfully demonstrated to detect the viral particles in a sample at a range of concentrations, including 10\hat{ }3 particles/mL.

### Example 2: Artificial Biopolymer Complex Used to Detect Virus Concentrations in Samples with and without Saliva

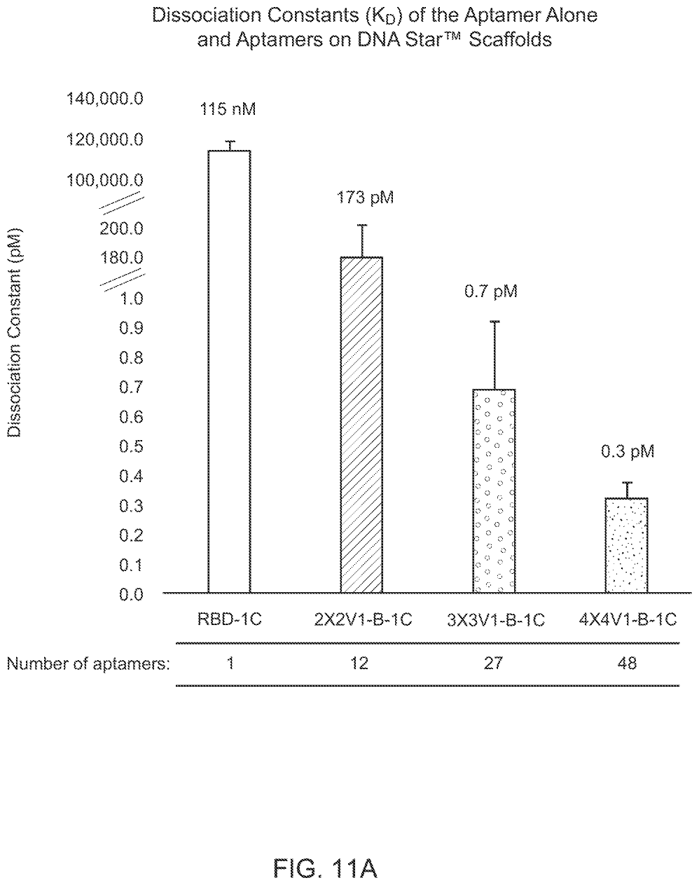

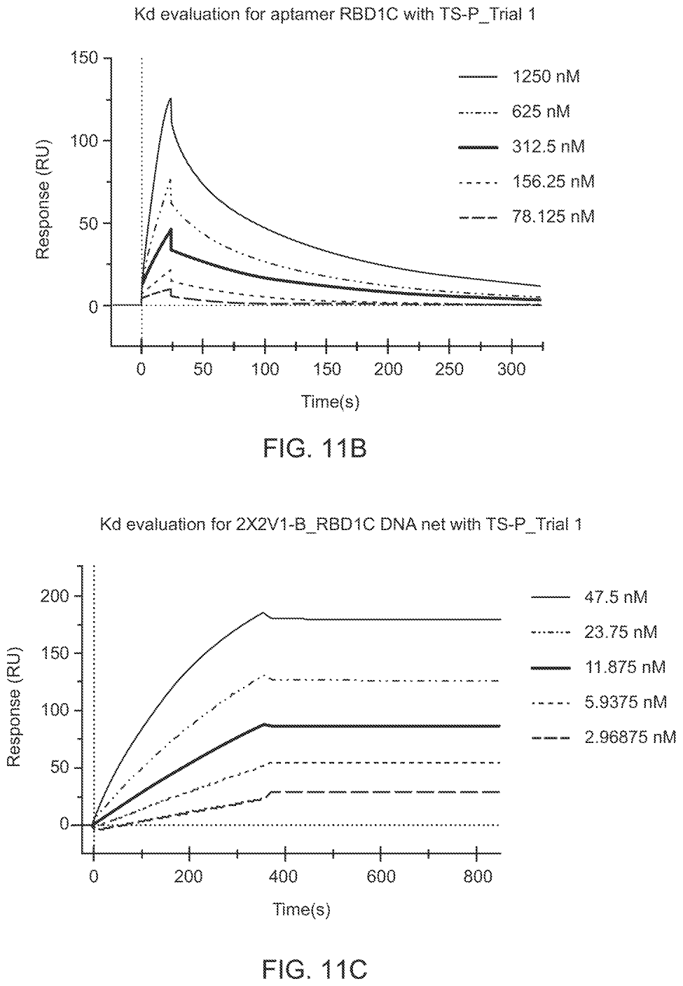

FIG. 10A shows the results of a fluorescence-based assay using a network of polynucleotides designed to bind to SARS-CoV-2 virions in a saliva sample at different virus concentrations. FIG. 10B shows the results of a fluorescence-based assay using a network of polynucleotides designed to bind to SARS-CoV-2 virions in a control sample at different virus concentrations. As shown in FIGS. 10A and 10B, the artificial biopolymer complex can be used to detect the virus at a wide range of copies/mL in samples both with and without saliva.

### Example 3: SPR Analysis of DNA STAR™ Binding to Immobilized SARS-CoV-2 Trimeric Spike Protein

Purified wild-type SARS-CoV-2 Trimeric Spike Protein (Meridian Bioscience, Ohio, USA) was immobilized onto a research grade CM5 S-Series SPR chip (GE healthcare, Uppsala, Sweden) according to a standard amine coupling protocol. Briefly, carboxymethyl groups on the CM5 chip surface in Flow Cells 1 and 2 were activated using a 420-second injection pulse at a flow rate 5 μL/min using a 4:1 mixture of N-ethyl-N-(dimethylaminopropyl) carbodiimide (EDC) and N-hydroxysuccinimide (NETS), respectively (final concentration of 200 mM EDC and 50 mM NHS, mixed immediately before injection). Following the activation, a 50 μg/mL Purified Mouse IgG (ImmunoReagents, North Carolina, USA) solution was prepared in a 10 mM sodium acetate (pH 5.0) buffer and then injected over the activated biosensor surface of Flow Cell 1. The successful immobilization of the Purified Mouse IgG was confirmed by the observation of a ˜6529 resonance unit (RU) increased baseline signal. Following the activation, a 50 μg/mL SARS-CoV-2 Trimeric Spike Protein was prepared in a 10 mM sodium acetate (pH 5.0) buffer and injected over the activated biosensor surface of Flow Cell 2. The successful immobilization of the Trimeric Spike Protein was confirmed by the observation of a 6629 resonance unit (RU) increased baseline signal. Excess unreacted carboxymethyl groups on the sensor surface were deactivated with a 600-second injection of 1 M ethanolamine in Flow Cells 1 and 2 at a flow rate 5 μL/min.

Flow Cell 1 with immobilized Purified Mouse IgG served as a reference for Flow Cell 2 with immobilized Trimeric Spike Protein. Different dilutions of each DNA-aptamer and DNA-Net-Aptamer complex were injected over the sensor chip at a flow rate of 5 μL/min with HBST-Mg, pH 7.4 (20 mM HEPES, 150 mM NaCl, 5 mM MgCl2, 0.05% Tween-20) as running buffer. At the end of the sample injection, the HBST-Mg, pH 7.4 was flowed over the sensor surface to facilitate dissociation. After a 5 min dissociation for the DNA-Aptamer and a 10 min dissociation time for DNA-Net-Aptamer complex, the sensor surface was fully regenerated by injecting 10 mM Glycine, pH 2 buffer for 30 second at a flow rate of 30 μL/min. The SPR response (sensorgram) was monitored as a function of time at 25° C. SPR measurements were performed on a BIAcore T200 (GE healthcare, Uppsala, Sweden) operated using the BIAcore T200 control software.

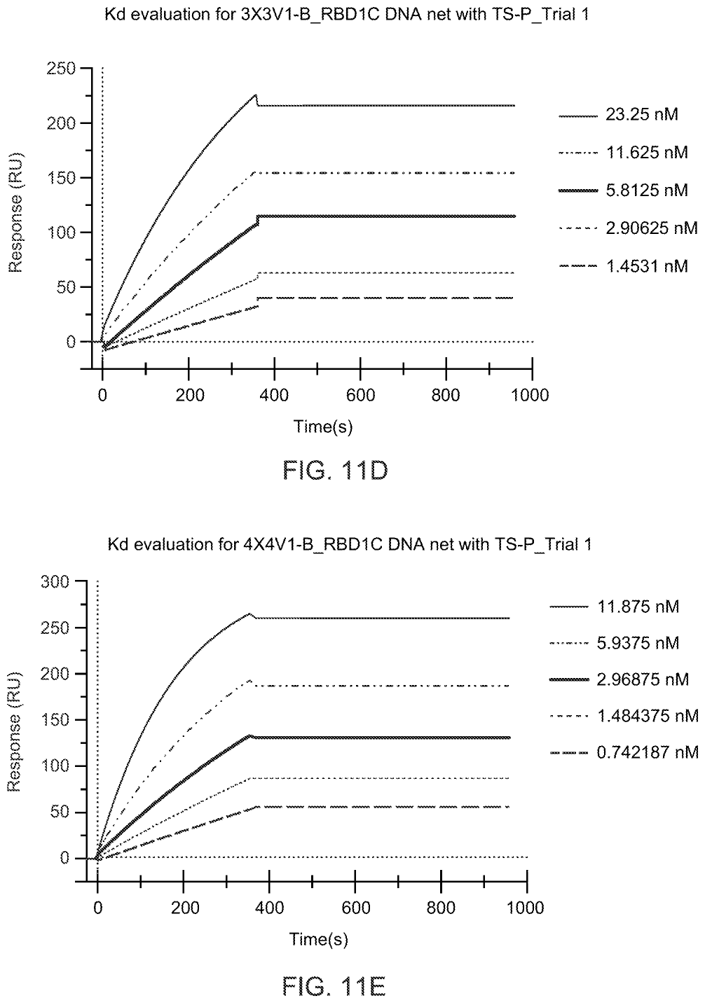

The resulting sensorgrams were used for binding kinetics parameter determination (i.e. association rate constant: ka; dissociation rate constant: kd; and binding equilibrium dissociation constant: K_{D}, K_{D}=kd/ka) by locally fitting the entire association and dissociation phases using 1:1 Langmuir binding model from BiaEvaluation software 4.0.1 (GE healthcare, Uppsala, Sweden). FIG. 11A shows dissociation constants (K_{D}) of aptamer alone and aptamer on DNA STAR™ scaffolds. FIG. 11B shows the K_{D} evaluation for the DNA-Aptamer with the SARS-CoV-2 Trimeric Spike Protein (TS-p). FIG. 11C shows the KD evaluation for a 2×2 DNA-Net-Aptamer complex with the SARS-CoV-2 Trimeric Spike Protein (TS-p). FIG. 11D shows the KD evaluation for a 3×3 DNA-Net-Aptamer complex with the SARS-CoV-2 Trimeric Spike Protein (TS-p). FIG. 11E shows the KD evaluation for a 4×4 DNA-Net-Aptamer complex with the SARS-CoV-2 Trimeric Spike Protein (TS-p).

### Diagnostic and Therapeutic Techniques

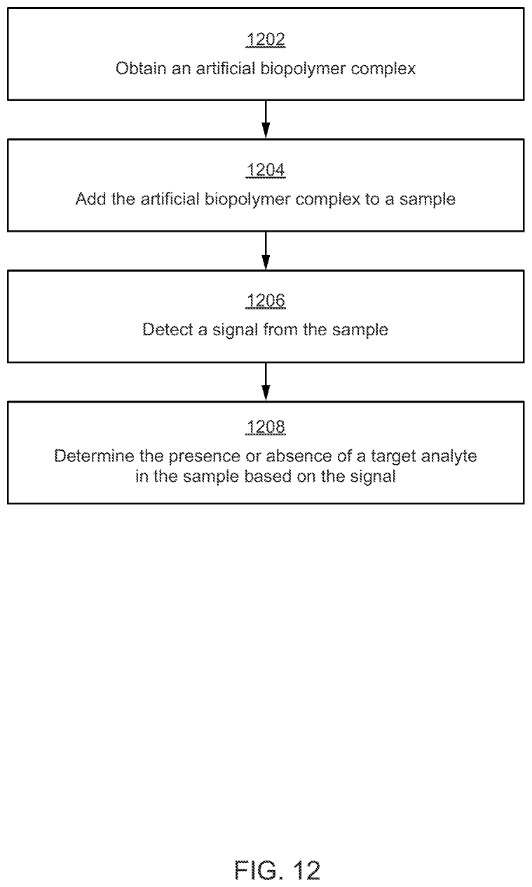

FIG. 12 illustrates a process for determining a presence or absence of a target analyte in a sample using an artificial biopolymer complex according to various embodiments of the present disclosure. The artificial biopolymer complex may be any of the artificial biopolymer complexes described with respect to FIGS. 1-11E.

At block 1202, an artificial biopolymer complex is obtained. The artificial biopolymer complex may be obtained based on the desired type of target analyte to be detected for a given subject. As used herein, when an action is “based on” something, this means the action is based at least in part on at least a part of the something. The artificial biopolymer complex may comprise a network of polynucleotides and sets of binders attached to the network of polynucleotides. The binders bind to antigens of the target analyte, and are attached at loci on one or more of the arms of the network of polynucleotides. The loci are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte. In certain instances, the binders are arranged in sets of clustered binders, and each binder of a set of clustered binders is attached to one of the three or more arms that form a junction. The binders of each of the sets of clustered binders may be attached to the arms at loci that are a predetermined distance from the junction, where the loci are separated by predetermined intra-binder distances such that each set of clustered binders are positioned on the network of polynucleotides in a predetermined two-dimensional or three-dimensional spatial pattern that matches a two-dimensional or three-dimensional spatial pattern of the one or more epitopes on an antigen. Each of the binders can be an aptamer, an antibody, a peptide, a nanobody, an antibody mimic (e.g., an affimer or a molecularly imprinted polymer), or a small analyte ligand. Each antigen of the two or more antigen may be a different antigen, some may be the same and some may be different, or all of the antigens of the two or more antigens may be the same.

At block 1204, the artificial biopolymer complex is added to a sample. The sample may be a biological sample such as sputum/saliva. The sample may include the target analyte, such as SARS-CoV-2. At least some of the binders on the network of polynucleotides can bind to epitopes on antigens of the target analyte. The binding of the network of polynucleotides to the target analyte may trigger the release of a signal from the network of polynucleotides. For example, in response to the binders binding to the target analyte, one or more quenchers may be released from an attachment to the locking molecules, the network of polynucleotides, or the binders. The release of the quenchers can allow for the generation of a fluorescent signal by one or more fluorophores that were previously quenched by the one or more quenchers. Alternatively or additionally, in response to the binders binding to the target analyte, a conformation of the binders attached to the antigens or the epitopes of the antigens of the target analyte may be changed. The conformation change to the binders may reduce quenching of a fluorescent signal caused by one or more of the quenchers. Reducing the quenching by the one or more quenchers may cause the fluorophores to generate a fluorescent signal.

At block 1206, a signal is detected from the sample. For example, if the binders or locking molecules on the network of polynucleotides comprise a fluorophore, the signal may be a fluorescence signal. In some instances, the signal may be detected over a detection period of time to identify a rate of change of the signal during the detection period of time. For example, the detection period of time may be 100 seconds in length or between 30 seconds to 10 minutes in length. In some instances, the detection period may be after an initial incubation period. For example, the initial incubation period may be from 5 to 200 seconds after adding the artificial biopolymer complex to the sample.

At block 1208, the presence or absence of the target analyte in the sample is determined based on the signal. The determination may be a qualitative or quantitative determination based on the signal. In some instances, the rate of change of the signal during the detection period of time may be used to quantitatively determine the presence or absence of the target analyte. For example, the presence of the target analyte may be determined if the rate of change is above a certain threshold. The rate of change required to meet the threshold can depend on factors relevant to the assay, including desired sensitivity, specificity, and efficiency. In some instances, the rate of change in the signal is 5%, 10%, 15%, 25%, 30%, 35%, 40%, 45%, or 50% or greater. Alternatively or additionally, the presence of the target analyte can be qualitatively determined based on visual observation of the fluorescence signal.

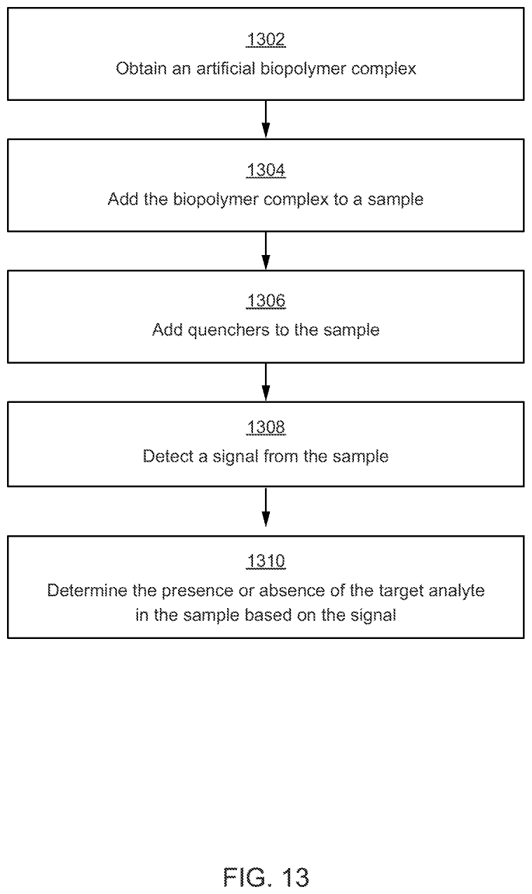

FIG. 13 illustrates a process for determining a presence or absence of a target analyte in a sample using an artificial biopolymer complex according to various embodiments of the present disclosure. The artificial biopolymer complex may be any of the artificial biopolymer complexes described with respect to FIGS. 1-11E.

At block 1302, an artificial biopolymer complex is obtained. The artificial biopolymer complex may be obtained based on the desired type of target analyte to be detected for a given subject. In some instances, the artificial biopolymer complex further comprises fluorophores attached to the locking molecules, the network of polynucleotides, or the binders. The artificial biopolymer complex may not initially comprise quenchers. At this stage, the fluorescent signal from the fluorophores may be detected and a control reading of the fluorescent signal may be obtained (e.g., control reading in relative fluorescence units (RFU)).

At block 1304, the artificial biopolymer complex is added to a sample. The sample may be a biological sample such as sputum/saliva. The sample may include the target analyte, such as SARS-CoV-2. At least some of the binders on the network of polynucleotides can bind to the target analyte. For example, the binders may bind to antigens of the target analyte or to the epitopes of antigens of the target analyte. Some of the networks of polynucleotides may become fully saturated with antigen (i.e., substantially all binders have bound to the antigen). Some of the networks of polynucleotides may become partially saturated with antigen (i.e., a smaller percentage, e.g., less than 50%, of the binders have bound to the antigen).

At block 1306, quenchers are added to the sample. The quenchers may be attached to oligonucleotides or polynucleotides that are structured to attach to the binders. The quenchers may bind to one or more binders that are not attached to the antigens of the target analyte or the epitopes of the antigens of the target analyte. By binding to the binders, the quenchers may quench the fluorescent signal emitted by one or more fluorophores attached to the locking molecules, the network of polynucleotides, or the binders. In some instances, the sample may be incubated with the artificial biopolymer complex for a first predetermined amount of time before adding the quenchers to the sample. For example, the first predetermined amount of time may be 30 minutes in length. The sample and artificial biopolymer complex may be incubated with orbital shaking. In some instances, after incubating for the first predetermined amount of time, but before adding the quenchers to the sample, the fluorescent signal from the fluorophores may be detected and a base test reading of the fluorescent signal may be obtained (e.g., a first reading in RFU).

At block 1308, a signal is detected from the sample. In some instances, the fluorescent signal from the fluorophores may be detected and a test reading of the fluorescent signal may be obtained (e.g., a second reading in RFU). In some instances, prior to detecting the signal from the sample, the sample is incubated with the artificial biopolymer complex and the quenchers for a second predetermined amount of time. For example, the second predetermined amount of time may be 60 seconds. In other instances, the signal is detected over a detection period of time to identify a rate of change of the signal during the detection period of time. For example, a signal from the fluorophores may be detected and a test reading of the fluorescent signal may be obtained after the second predetermined amount of time and after a third predetermined amount of time. For example, the third predetermined amount of time may be 75 seconds. Thereafter, the rate of change of the signal during the detection period of time is determined based on the readings.

At block 1310, the presence or absence of the target analyte in the sample is determined based on the signal. In some instances, the presence or absence of the target analyte in the sample may be based on the control reading, the first reading, the second reading, or any combination thereof. For example, because the quenchers may bind to one or more binders that are not bound to the target analyte and thus quench signals released from fluorophores attached to one or more binders, the second reading may be a more accurate representation of the amount of antigen present in the sample than the first reading. In instances where the signal is detected over a detection period of time to identify a rate of change of the signal during the detection period of time, the rate of change above a threshold may be indicative of the absence of the target analyte. For example, samples that include less antigen or no antigen may have signals that are quenched more rapidly and completely than samples that include more or some antigen, and thus the rate of change may be above a threshold and indicative of the absence of the target analyte.

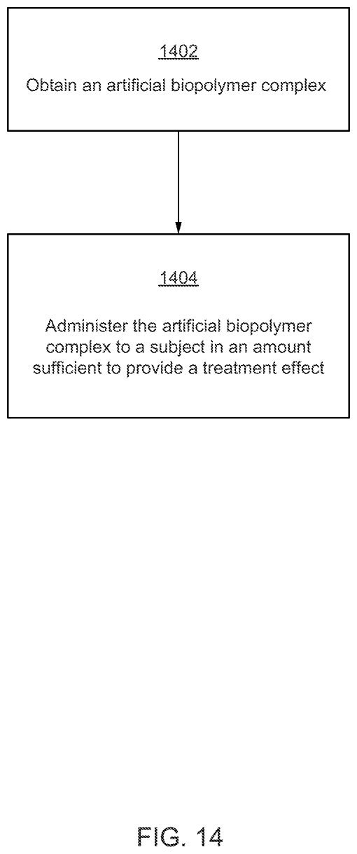

FIG. 14 illustrates a process for treating a subject using an artificial biopolymer complex according to various embodiments of the present disclosure. The artificial biopolymer complex may be any of the artificial biopolymer complexes described with respect to FIGS. 1-13.

At block 1402, an artificial biopolymer complex is obtained. The artificial biopolymer complex may be obtained based on the desired type of treatment to be provided for a given subject. In some instances, the artificial biopolymer complex further comprises one or more therapeutic agents attached to the network of polynucleotides. For example, the artificial biopolymer complex may be designed to carry a therapeutic agent via ligand binding to surface antigens. Therapeutic agent means a drug, protein, peptide, gene, compound or other pharmaceutically active ingredient that can be used in the application of chemotherapy, antibody therapy, immunotherapy, immunization, or the like for the treatment or mitigation of a disease condition or ailment.

At block 1410, the artificial biopolymer complex is administered to the subject in an amount sufficient to provide a treatment effect. In some instances, the treatment effect is a prophylactic effect or a therapeutic effect. The treatment effect is facilitated by binding of the binders to the antigens. In some instances, the binding of the binders to the antigens activates a response by the target analyte to the artificial biopolymer complex. The response may be an ingestion of the artificial biopolymer complex by the target analyte. The ingestion of the artificial biopolymer complex by the target analyte may cause the release of the one or more therapeutic agents from the network of polynucleotides.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

The present disclosure includes embodiments in which exactly one member of the group is present in, employed in, or otherwise relevant to a given product or process.

The present disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference for all purposes, even if not expressly stated in the citation. In case of conflicting statements of a cited source and the instant application, the statement in the instant application shall control.

Section and table headings are not intended to be limiting.

1. An artificial biopolymer complex comprising:
a network of polynucleotides comprising structural units connected to one another via a series of arms and junctions, wherein:
each of the structural units have a predetermined shape defined by one or more strands of polynucleotides;
at least a portion of the one or more strands of polynucleotides of each structural unit is complementary to at least a portion of the one or more strands of polynucleotides of another structural unit, and the complementary portions of the strands of the polynucleotides are hybridized to connect the structural units;
the complementary portions of the strands of the polynucleotides form the arms with a predetermined length; and
intersections of three or more arms form the junctions at a predetermined distance from one another based on the predetermined length of the arms; and
binders attached to the network of polynucleotides, wherein:
the binders bind to antigens of a target analyte; and
the binders are attached at loci on one or more of the arms forming the junctions, wherein the loci are separated by predetermined inter-binder distances such that the binders are positioned on the network of polynucleotides in a predetermined two-dimensional or three-dimensional spatial pattern that matches a two-dimensional or three-dimensional spatial pattern of the antigens on the target analyte.

2. The artificial biopolymer complex of claim 1, wherein:
each of the antigens comprises one or more epitopes;
the binders are arranged in sets of clustered binders;
each binder of a set of clustered binders is attached to one of the three or more arms that form a junction; and
the binders of each of the sets of clustered binders are attached to the arms at loci that are a predetermined distance from the junction, wherein the loci are separated by predetermined intra-binder distances such that each set of clustered binders are positioned on the network of polynucleotides in a predetermined two-dimensional or three-dimensional spatial pattern that matches a two-dimensional or three-dimensional spatial pattern of the one or more epitopes on an antigen.

3. The artificial biopolymer complex of claim 1, wherein the junctions are formed by at least 2N arms extending therefrom, and wherein N is at least 2.

4. The artificial biopolymer complex of claim 1, wherein each of the junctions are formed by at least N arms extending therefrom, and wherein N is at least 3.

5. The artificial biopolymer complex of claim 1, wherein N binders are attached to the arms that form each of the junctions, and wherein N is at least 1.

6. The artificial biopolymer complex of claim 5, wherein N is at least 2, and wherein the N binders are attached to alternating arms that form each of the junctions.

7. The artificial biopolymer complex of claim 1, wherein the two-dimensional or three-dimensional spatial pattern of the antigens is defined by intermolecular spacing of the antigens on a surface of the target analyte.

8. The artificial biopolymer complex of claim 7, wherein the predetermined inter-binder distances of the loci of the binders match the intermolecular spacing of the antigens such that the binders align spatially with the antigens on the surface of the target analyte.

9. The artificial biopolymer complex of claim 7, wherein:
each of the antigens is (i) a length and width in angstroms or nanometers from other antigens on the target analyte or (ii) a length, width, and depth in angstroms or nanometers from the other antigens on the target analyte, which define the intramolecular spacing of the antigens on the target analyte;
each of the binders is (i) a length and width in angstroms or nanometers from other binders on the network of polynucleotides or (ii) a length, width, and depth in angstroms or nanometers from the other binders on the network of polynucleotides, which defines the predetermined inter-binder distances of the loci of the binders; and
the predetermined inter-binder distances of the loci of the binders match the intermolecular spacing of the antigens such that the binders align spatially with the antigens on the surface of the target analyte.

10. The artificial biopolymer complex of claim 2, wherein the two-dimensional or three-dimensional spatial pattern of the one or more epitopes is defined by intramolecular spacing of the one or more epitopes on a surface of the antigen.

11. The artificial biopolymer complex of claim 10, wherein the predetermined intra-binder distances of the loci of the binders of each of the sets of clustered binders match the intramolecular spacing of the one or more epitopes such that the sets of clustered binders align spatially with the one or more epitopes on the surface of the antigens.

12. The artificial biopolymer complex of claim 10, wherein:
each of the epitopes is (i) a length and width in angstroms or nanometers from other epitopes on the antigen or (ii) a length, width, and depth in angstroms or nanometers from the other epitopes on the antigen, which define the intramolecular spacing of the one or more epitopes on the antigen;
each of the binders of each of the sets of clustered binders is (i) a length and width in angstroms or nanometers from other binders of each of the sets of clustered binders on the network of polynucleotides or (ii) a length, width, and depth in angstroms or nanometers from the other binders of each of the sets of clustered binders on the network of polynucleotides, which defines the predetermined intra-binder distances of the loci of the binders of each of the sets of clustered binders; and
the predetermined intra-binder distances of the loci of the binders of each of the sets of clustered binders match the intermolecular spacing of the epitopes such that each of the sets of clustered binders align spatially with the one or more epitopes on the surface of the antigens.

13. The artificial biopolymer complex of claim 1, wherein each of the structural units have the same predetermined shape defined by the one or more strands of polynucleotides.

14. The artificial biopolymer complex of claim 13, wherein the network of polynucleotides has a length L and a width W defined by a number S of structural units, and wherein L is 1 or more and W is 1 or more.

15. The artificial biopolymer complex of claim 14, wherein L is 2 between 2 and 5 and W is between 2 and 5.

16. The artificial biopolymer complex of claim 14, wherein the predetermined shape is a rhombus, a triangle, a pentagon, or a hexagon.

17. The artificial biopolymer complex of claim 1, wherein the one or more strands of polynucleotides are single stranded DNA, and the arms are double stranded DNA.

18. The artificial biopolymer complex of claim 1, wherein the target analyte is severe acute respiratory syndrome coronavirus 2 (SARS-CoV-2), and the antigens comprise trimeric spike glycoproteins.

19. The artificial biopolymer complex of claim 1, wherein each of the binders is an aptamer, an antibody, a peptide, a nanobody, an antibody mimic, or a small analyte ligand.

20. The artificial biopolymer complex of claim 1, further comprising locking molecules that attach each of the binders to the network of polynucleotides.

21. The artificial biopolymer complex of claim 20, wherein the locking molecules comprise a single stranded chain of nucleic acids hybridized to form a portion of the arms attached to the binders.

22. The artificial biopolymer complex of claim 20, further comprising quenchers attached to the locking molecules and fluorophores attached to the binders.

23. The artificial biopolymer complex of claim 20, further comprising quenchers attached to the locking molecules, the locking molecules are bound to molecules, and fluorophores attached to the binders.

24. The artificial biopolymer complex of claim 20, further comprising quenchers attached to the binders and fluorophores attached to the locking molecules.

25. The artificial biopolymer complex of claim 1, further comprising (i) quenchers attached to the network of polynucleotides and fluorophores attached to the binders, or (ii) quenchers attached to the binders and fluorophores attached to the network of polynucleotides.

26. The artificial biopolymer complex of claim 1, further comprising quenchers and fluorophores attached to the binders.

27. A method for determining a presence or absence of a target analyte in a sample, the method comprising:
obtaining the artificial biopolymer complex of claim 1;
adding the artificial biopolymer complex to the sample;
detecting a signal from the sample; and
determining the presence or absence of the target analyte in the sample based on the signal.

28. The method of claim 27, wherein the determining is a qualitative or quantitative determination based on the signal.

29. The method of claim 27, wherein the signal is detected over a detection period of time to identify a rate of change of the signal during the detection period of time, and wherein the rate of change above a threshold is indicative of the presence of the target analyte.

30. The method of claim 29, wherein the detection period of time is about 100 seconds in length.

31. The method of claim 29, wherein the detection period of time is from about seconds to 10 minutes in length.

32. The method of claim 27, wherein the signal is a fluorescent signal.

33. The method of claim 32, further comprising:
binding the artificial biopolymer complex to the target analyte;
in response to the binding, releasing one or more of the quenchers from the locking molecules, the network of polynucleotides, or the binders; and
in response to the release of the one or more quenchers, generating the fluorescent signal by one or more fluorophores that are no longer quenched by the one or more quenchers.

34. The method of claim 32, further comprising:
binding the artificial biopolymer complex to the target analyte;
in response to the binding, changing a conformation of the binders attached to the antigens of the target analyte or the epitopes of the antigens of the target analyte;
in response to the conformation change to the binders, reducing quenching of the fluorescent signal by one or more of the quenchers; and
in response to reducing the quenching, generating the fluorescent signal by one or more fluorophores that are no longer quenched by the one or more quenchers.

35. A method for determining a presence or absence of a target analyte in a sample, the method comprising:
obtaining the artificial biopolymer complex of claim 1;
adding the artificial biopolymer complex to the sample;
adding quenchers to the sample;
detecting a signal from the sample; and
determining the presence or absence of the target analyte in the sample based on the signal.

36. The method of claim 35, wherein:
the quenchers are attached to oligonucleotides structured to attach to the binders;
fluorophores are attached to the locking molecules, the network of polynucleotides, or the binders; and
the signal is a fluorescent signal.

37. The method of claim 36, further comprising:
binding the artificial biopolymer complex to the target analyte;
binding the quenchers to one or more binders that do not attach to the antigens of the target analyte or the epitopes of the antigens of the target analyte; and
in response to the binding of the quencher, quenching the fluorescent signal by one or more fluorophores attached to the locking molecules, the network of polynucleotides, or the binders.

38. The method of claim 37, further comprising:
prior to adding the quenchers to the sample, incubating the sample with the artificial biopolymer complex for a first predetermined amount of time;
after the incubating for the first predetermined amount of time and prior to adding the quenchers to the sample, detecting the signal from the sample to obtain a first reading; and
prior to detecting the signal from the sample, incubating the sample with the artificial biopolymer complex and the quenchers for a second predetermined amount of time,
wherein the detecting the signal from the sample after adding the quenchers obtains a second reading, and the presence or absence of the target analyte in the sample is determined based on the first reading and the second reading.

39. The method of claim 38, wherein the signal is detected over a detection period of time to identify a rate of change of the signal during the detection period of time, and wherein the rate of change above a threshold is indicative of the absence of the target analyte.

40. A method for treating a subject, the method comprising:
obtaining the artificial biopolymer complex of claim 1; and
administering the artificial biopolymer complex to the subject in an amount sufficient to provide a treatment effect.

41. The method of claim 40, wherein the treatment effect is a prophylactic effect or a therapeutic effect.

42. The method of claim 40, wherein the artificial biopolymer complex further comprises one or more therapeutic agents attached to the network of polynucleotides.
